(19) United States
(12) Patent Application Publication
(10) Pub. No.: US 2005/0095663 A1
(43) Pub. Date: May 5, 2005

(54) Process for screening fungitoxic compounds

(76) Inventors: David Hamilton Young, Carmel, IN (US); Richard A. Slawecki, Warminster, PA (US); Ronald Ross Jr, Zionsville, IN (US)

Correspondence Address: DOW AGROSCIENCES LLC, 9330 ZIONSVILLE RD, INDIANAPOLIS, IN, 46268, US

(21) Appl. No.: 10/486,524

(22) PCT Filed: Jun. 17, 2003
(86) PCT No.: PCT/US03/19158

Related U.S. Application Data
(60) Provisional application No. 60/389,325, filed on Jun. 17, 2002.

(51) Int. Cl. C12Q 1/26; A01N 37/00

(52) U.S. Cl. 435/25; 514/560

(57) ABSTRACT

Methods for identifying fungitoxic compounds are disclosed based on inhibition of the enzyme Δ-9 fatty acid de-saturase.

This application claims priority from U.S. provisional application No. 60/389,325, which was filed on Jun. 17, 2002.

The present invention relates to the surprising observation that a class of pyridazinone fungicides, known to be effective in controlling a wide range of fungal diseases on plants without adverse effects on the plants, exert their fungitoxic effect by inhibition of a fungal Δ-9 fatty acid desaturase enzyme. More particularly, the invention relates to a method for identifying fungitoxic compounds by evaluating potential candidates for inhibition of Δ-9 fatty acid desaturase activity and using the inhibitors of Δ-9 fatty acid desaturase as fungicides.

In fungi and mammals, monounsaturated fatty acids are generated from saturated fatty acyl-CoA substrates by a membrane-bound enzyme system involving Δ-9 fatty acid desaturase, cytochrome b₅ and NADH-dependent cytochrome b₅ reductase in a reaction which requires oxygen {Bloomfield D. K. and Bloch, K, The Journal of Biological Chemistry 235, 337-345 (1960), Jeffcoat, R., Essays in Biochemistry 15, 1-36 (1979)). Comparison of the primary amino acid sequence of the Δ-9 fatty acid desaturase enzymes in rat and yeast show substantial homology between the mammalian and fungal enzymes {Stukey, J. E., McDonough, V. M. and Martin, C. E. The Journal of Biological Chemistry 265, 20144-20149 (1960)}. Cyclopropenoid fatty acids, such as the plant-derived material sterculic acid, are known inhibitors of Δ-9 fatty acid desaturase in animals and fungi, and in both types of organism produce profound changes in fatty acid composition characterized by higher ratios of saturated to unsaturated fatty acids {(Christie, W. W. in Topics in Lipid Chemistry Vol. 1, pp. 1-49, ed. by F. D. Gunstone (1970), Moreton, R. S. Applied Microbiology and Biotechnology 22, 41-45 (1985)}. Sterculic acid is also reported to inhibit unsaturated fatty acid biosynthesis in plants {James, A. T., Harris, P. and Bezard, J., European Journal of Biochemistry 3, 318-325 (1968)}. The possibility that cyclopropenoid fatty acids might serve an antifungal role in plants has been discussed by Schmid and Patterson {(Lipids 23, 248-252 (1988)}. These authors showed that cyclopropenoid fatty acids at 30 μM caused a partial inhibition of growth of the plant pathogens Rhizoctonia solani and Ustilago maydis in culture, but no growth inhibition of Fusarium oxysporum. The unsaturated fatty acid, oleic acid, prevented inhibition of growth by cyclopropenoid fatty acids, and in discussing a possible antifungal role of cyclopropenoid fatty acids these authors suggested that the fatty acid background in which cyclopropenoid fatty acids occur would be important. In other words, sterculic acid might not be capable of inhibiting fungi in plants due to the presence in plants of unsaturated fatty acids, which are major cellular components. The influence of the unsaturated fatty acid environment is also evidenced by the fact that in the yeast Saccharomyces cerevisiae the Δ-9 fatty acid desaturase enzyme is not required for growth if a supply of unsaturated fatty acids is present {Stewart, L. C. and Yaffe, M. P., Journal of Cell Biology 115, 1249-1257 (1991)}. In fungal yeasts, Moreton disclosed in Applied Microbiology and Biotechnology 22, 41-45 (1985) that cyclopropenoid fatty acids caused dramatic increases in the ratio of saturated to unsaturated fatty acids in Candida sp. 107, Trichosporon cutaneum and Rhodosporidium toruloides without significant effects on growth. It is important to highlight that there is no data in the prior art which shows efficacy of cyclopropenoid fatty acids in controlling fungal growth for plants or animals

Although Δ-9 fatty acid desaturase is a well-studied enzyme and the cyclopropenoid fatty acids are known inhibitors of this enzyme, it is not obvious from the above mentioned prior art that this enzyme would be a good biochemical target for screening potential fungicides. Indeed, various disclosures would argue against the Δ-9 fatty acid desaturase enzyme system being a good biochemical target. In culture, cyclopropenoid fatty acids did not inhibit growth of various fungal yeasts despite producing a considerable shift in fatty acid composition as described by Moreton in Applied Microbiology and Biotechnology 22, 41-45 (1985). In addition, it has not been shown that cyclopropenoid fatty acids can control fungal infections of plants or animals, and the fact that unsaturated fatty acids are present in large amounts in lipids from both plants and animals would suggest a fatty acid environment in plants and animals in which an inhibitor of Δ-9 fatty acid desaturase might be ineffective. Furthermore, the structural similarity between the mammalian and fungal enzymes and the fact that cyclopropenoid fatty acids exert a major effect on the ratio of saturated to unsaturated fatty acids in animals and plants would imply a lack of selectivity of potential enzyme inhibitors and suggest the likelihood of undesirable toxicological effects on mammals and adverse effects on plants.

Surprisingly, the inventors have discovered that a class of pyridazinone fungicides, known to be effective in controlling a wide range of fungal diseases on plants without adverse effects on the plants, exert their fungitoxic effect by inhibition of a fungal enzyme Δ-9 fatty acid desaturase enzyme. Furthermore, the inventors have found that these compounds can control fungal infection in a mammalian system and do not inhibit activity of a mammalian Δ-9 fatty acid desaturase enzyme. These findings indicate that fungal Δ-9 fatty acid desaturases are highly attractive biochemical targets for screening potential fungicides. Thus compounds designed to inhibit fungal Δ-9 fatty acid desaturases, or compounds selected for their ability to inhibit fungal Δ-9 fatty acid desaturases through screening of synthetic chemical libraries or libraries of natural products have the potential to be commercially useful fungicides.

In the presence of an inhibitor of Δ-9 fatty acid desaturase, fungal cells have a higher ratio of saturated to unsaturated fatty acids in their membranes. Resulting changes in membrane stability or fluidity are presumed to cause fungitoxicity due to impaired membrane function. A unique characteristic of the pyridazinone fungicides is the ability of saturated fatty acids to enhance their fungitoxicity as disclosed by Young et al in U.S. Pat. No. 5,741,793. While not being limited by theory, enhancements in fungitoxicity of Δ-9 fatty acid desaturase inhibitors are expected from the addition of exogenous saturated fatty acids by promoting a shift to higher ratios of saturated to unsaturated fatty acids in fungal membranes.

Accordingly, one aspect of the present invention is a method for identifying potential fungicides for controlling diseases of plants or mammals or controlling fungal growth on a substrate, which involves testing one or more candidate compounds in an assay which detects the inhibition of Δ-9 fatty acid desaturase enzyme activity and subsequently subjecting the compounds which inhibit the enzyme to one or more conventional tests to confirm fungicidal activity.

A second aspect of the present invention is a method for detecting compounds which inhibit Δ-9 fatty acid desaturase activity including: (a) testing a candidate compound for fungitoxicity in the presence and absence of an exogenous supply of an unsaturated fatty acid and a saturated fatty acid; and (b) identifying inhibitors of Δ-9 fatty acid desaturase by demonstrating reduced fungitoxicity in the presence of the exogenous supply of unsaturated fatty acid and no reduction in fungitoxicity or enhanced fungitoxicity in the presence of the exogenous supply of saturated fatty acid.

A third aspect of the present invention provides a composition having synergistic fungitoxic effects which includes (a) one or more fungitoxic Δ-9 fatty acid desaturase inhibitors; and (b) one or more saturated fatty acids.

A fourth aspect of the present invention provides a fungitoxic formulation including (a) one or more Δ-9 fatty acid desaturase inhibitors; (b) one or more fungicides that are not Δ-9 fatty acid desaturase inhibitors; (c) one or more carriers; (d) optionally, one or more additives; and optionally (e) one or more saturated fatty acids.

The inventors have demonstrated that a class of pyridazinone fungicides, known to be effective in controlling a wide range of fungal diseases on plants without adverse effects on the plants (European Patent Publication No. EP 478 195 A1), exert their fungitoxic effect by inhibition of the fungal enzyme Δ-9 fatty acid desaturase. Pyridazinone fungicides have been disclosed by Young et al in U.S. Pat. No. 5,741,793. Pyridazinones usefully employed in accordance with the present invention were prepared according to the methods described in detail in U.S. Pat. No. 5,753,642.

Suitable examples of pyridazinones include, but are not limited to, 6-(4-chlorophenyl)-2-(2′-pentyn-4′-ene-1-yl)-3(2H)-pyridazinone; 6-(4-chloro-phenyl)-2-(2′-pentynyl)-3(2H)-pyridazinone; 6-(4-chlorophenyl)-2-(5′-pentoxy-2′-butynyl)-3(2H)-pyridazinone; 6-(4-chlorophenyl)-2-(4′-fluoro-2′-butynyl)-3(2H)-pyridazinone; 6-(2-pyridyl)-2-(2′-nonynyl)-3(2H)-pyridazinone; 7-chloro-2,4,4a,5-tetrahydro-2-(2′-pentynyl)-indeno[1,2-c]-pyridazin-3-one; 6-(4-chloro-phenyl)-2-(2′-pentynyl)-3(2H)-4,5-dihydropyridazinone; 6-(2-napthyl)-2-(2′-pentynyl)-3(2H)-pyridazinone; and 6-(4-chlorophenyl)-2-(2′-decynyl)-3(2H)-pyridazinone.

The inventors have discovered that these compounds can control fungal infection in a mammalian system (Example 1). Furthermore these compounds are potent inhibitors of the fungal Δ-9 fatty acid desaturase enzyme and do not inhibit the mammalian Δ-9 fatty acid desaturase enzyme (Example 2). The findings demonstrate that fungal Δ-9 fatty acid desaturases are highly attractive targets for the screening of potential fungicides. Compounds designed to inhibit fungal Δ-9 fatty acid desaturases, or compounds selected for their ability to inhibit fungal Δ-9 fatty acid desaturases through screening of synthetic chemical libraries or libraries of natural products have the potential to be commercially useful fungicides for controlling diseases of plants or mammals or controlling fungal growth on a substrate.

The first aspect of the present invention provides a method for identifying potential fungicides for controlling diseases of plants or mammals or controlling fungal growth on a substrate, which involves testing one or more candidate compounds in an assay which detects the inhibition of Δ-9 fatty acid desaturase enzyme activity. Subsequently, the compounds which bind to and inhibit the enzyme are subjected to one or more conventional tests to confirm fungicidal activity. The second step is not a requirement of the method of the invention. The present method for identifying potential anti-fungal compounds does not require the use of any particular Δ-9 fatty acid desaturase assay. While suitable assays are described herein (see examples 2 and 3) it is recognized that functionally equivalent assays can be substituted by those skilled in the art.

A separate embodiment of the present invention provides a method for detecting compounds which inhibit Δ-9 fatty acid desaturase activity which includes assaying the activity of Δ-9 fatty acid desaturase in a microsomal preparation from fungi in the absence and presence of candidate compounds. The activity of the enzyme is typically determined by measuring the conversion of a saturated fatty acyl-coenzyme A substrate to an unsaturated fatty acid product.

In order to obtain acceptable fungicidal activity using the conventional test(s) for fungitoxicity and the method of the present invention, a fungicidally effective amount of the composition must be used. As used herein, a “fungicidally effective amount” is a quantity of a compound which causes a reduction of a fungal population or decreases crop damage as compared to a control group. A fungicidally effective amount of a particular compound for use against a particular fungus will depend upon the type of equipment employed, the method and frequency of application desired, and the diseases to be controlled, but is typically from 0.01 to 20 kilograms (kg) of active compound per hectare. As a foliar fungicide, a pyridazinone is typically applied to growing plants at a rate of from 0.1 to 5, and preferably from 0.125 to 0.5 kg per hectare.

In a preferred embodiment, the present invention provides a method of identifying potential fungicides for controlling diseases of plants or mammals or controlling fungal growth on a substrate including but not limited to wood, leather, concrete, paints, plastics, metals and surfaces having a protective coating wherein the fungus belongs to the Ascomycete, Basidiomycete, Deuteromycete and Oomycete classes of fungi.

In another preferred embodiment, the present invention provides a method for identifying potential fungicides for control of fungal infections on plants which comprises testing a candidate compound in an assay which detects the inhibition of Δ-9 fatty acid desaturase activity in whole cells or cell extracts from a plant pathogenic fungus. Examples include but are not limited to: Colletotrichum spp., Magnaporthe spp., Botrytis spp., Fusarium spp., Alternaria spp., Helminthosporium spp., Venturia spp., Cercospora spp., Septoria spp., Mycosphaerella spp., Monilinia spp., Sclerotinia spp., Puccinia spp., Phytophthora spp., Pythium spp., Erysiphe spp., Penicillium spp. and Puccinia spp.

Another preferred embodiment of the present invention provides a method for identifying potential antifungal agents for controlling fungal infections in mammals which comprises testing a candidate compound in an assay which detects the inhibition of Δ-9 fatty acid desaturase activity in whole cells or cell extracts from a fungal pathogen of mammals. Examples include but are not limited to: Candida spp., Aspergillus spp., Fusarium spp., Coccidioides immitis, Cryptococcus neoformans, Histoplasma capsulatum, Microsporum spp., Tricophyton spp.

Yet another preferred embodiment of the present invention provides a method for identifying potential anti-fungal agents for controlling fungal growth which comprises testing a candidate compound in an assay which detects the inhibition of Δ-9 fatty acid desaturase in whole cells or cell extracts from Saccharomyces cerevisiae or other fungi which may serve as suitable model systems for plant or mammalian fungal pathogens.

A second aspect of the present invention is a method for detecting compounds which inhibit Δ-9 fatty acid desaturase activity including: (a) testing a candidate compound for fungitoxicity in the presence and absence of an exogenous supply of an unsaturated fatty acid and a saturated fatty acid; and (b) identifying inhibitors of Δ-9 fatty acid desaturase by demonstrating reduced fungitoxicity in the presence of the exogenous supply of unsaturated fatty acid and no reduction in fungitoxicity or enhanced fungitoxicity in the presence of the exogenous supply of saturated fatty acid (see Example 3).

A separate embodiment of the invention provides a method for controlling the growth of fungi which comprises applying a fungicidally effective amount of a Δ-9 fatty acid desaturase inhibitor to a locus where it is desired to control fungal growth. Useful fungicidal compositions are determined from the method of the present invention. For such purposes these compounds can be used in the technical or pure form as prepared, or more typically as solutions or as formulations. The compounds are usually taken up in a carrier or are formulated so as to render them suitable for subsequent dissemination.

When cells are treated with a toxic compound numerous physiological and biochemical changes result which may be characteristic of the mechanism of action of the compound. This may include changes in the amounts of particular metabolites and changes in the transcription of particular genes (treatment-responsive genes). The analysis of such changes can provide an indication of the likely mechanism of action of the compound. In the case of Δ-9 fatty acid desaturase inhibitors, such changes may include (but are not limited to) alterations in the levels of cellular fatty acids as shown in Example 4. Detection of changes in the transcription of treatment-responsive genes also provides a potential approach to identification of Δ-9 fatty acid desaturase inhibitors.

A third aspect of the present invention provides a composition having synergistic fungitoxic effects which includes (a) one or more fungitoxic Δ-9 fatty acid desaturase inhibitors; and (b) one or more saturated fatty acids.

Another characteristic of the pyridazinone fungicides is the ability of saturated fatty acids to enhance their fungitoxicity, as disclosed by Young et al in U.S. Pat. No. 5,741,793; the content and disclosure of which are usefully employed in the method of the present invention. In the presence of an inhibitor of Δ-9 fatty acid desaturase, fungal cells have a higher ratio of saturated to unsaturated fatty acids in their membranes. Resulting changes in membrane stability or fluidity presumably cause fungitoxicity due to impaired membrane function. While not being limited by theory, saturated fatty acids are expected to enhance fungitoxicity of Δ-9 fatty acid desaturase inhibitors by promoting this shift to a higher ratio of saturated to unsaturated fatty acids in fungal membranes.

Saturated fatty acid compounds suitable for use in the present invention have the formula CH₃(CH₂)ₙCO₂X, wherein X is selected from H, alkali metals and C₁-C₈ alkyl and wherein n is an integer selected from 8-22. The compounds are also referred to herein as “C₁₀-C₂₄ fatty acids and derivatives”, and as “fatty acids and derivatives”.

Suitable examples of salts and esters of C₁₀-C₂₄ fatty acids useful in increasing the effectiveness of a pyridazinone toward fungi are sodium salts and “short chain” alkyl esters, wherein X is selected from H, alkali metals and C₁-C₈ alkyl. As used herein, “short chain” refers to carbon chains of length C₁-C₈. The fatty acids and derivatives of the present invention may include hetero atoms such as oxygen, sulfur and nitrogen, and suitable fatty acids and derivatives may therefore include fatty acid ethers.

A fourth aspect of the present invention provides a fungitoxic formulation including (a) one or more Δ-9 fatty acid desaturase inhibitors; (b) one or more fungicides that are not Δ-9 fatty acid desaturase inhibitors; (c) one or more carriers; (d) optionally, one or more additives; and optionally (e) one or more saturated fatty acids. Separate embodiments of the present invention provide a fungicide formulation comprising a Δ-9 fatty acid desaturase inhibitor as the active ingredient in combination with or without a carrier. A separate embodiment provides a fungicide composition comprising Δ-9 fatty acid desaturase inhibitor in combination with one or more fungicides that are not Δ-9 fatty acid desaturase inhibitors.

Compounds determined to have fungitoxic activity from the method of the present invention can be used in a technical or pure form as prepared, or more typically as solutions or as formulations. The compounds are usually taken up in a carrier or are formulated so as to render them suitable for subsequent dissemination. The fungicidal formulations are effective in controlling fungal growth for plants or mammals or a substrate.

The fungicidal formulations of the present invention may be applied according to conventional methods for the use of fungicides, as described in U.S. Pat. No, 5,741793. The compounds determined as fungicides by the method of the invention, both the composition and formulation, may be applied separately, or may be combined to prepare the formulation before applying. As discussed herein, in most applications a fungicidal compound is used with an agronomically acceptable carrier. An “agronomically acceptable carrier” is a solid or liquid which is biologically, chemically and physically compatible with the compounds of the present invention, and which may be used in agricultural applications. Agronomically acceptable carriers suitable for use in the method of the present invention include organic solvents, and finely divided solids, both exemplified herein. For example, these fungicidal compositions can be formulated as wettable powders, emulsifiable concentrates, dusts, granular formulations, aerosols, or flowable emulsion concentrates. In such formulations, the compounds are extended with a liquid or solid carrier and, when desired, suitable surfactants are incorporated.

Optionally added components or additives, not required for fungicidal activity but useful or required for other properties, include, but are not limited to, adjuvants such as wetting agents, spreading agents, dispersing agents, stickers, adhesive and the like. Such adjuvants are well known in the art, and a discussion of adjuvants can be found in many references, such as in the John W. McCutcheon, Inc. publication MeCutcheon's Emulsifiers and Detergents (published annually by McCutcheon Division of MC Publishing Company, New Jersey).

In general, the fungitoxic compounds identified from the method of this invention may be dissolved in solvents such as acetone, methanol, ethanol, dimethylformamide, pyridine or dimethyl sulfoxide and such solutions can be diluted with water. The concentrations of the solution after dilution may vary from 1% to 90% by weight, with a preferred range being from 5% to 50%.

For the preparation of emulsifiable formulations and concentrates of the fingitoxic compounds of the present invention, the compound can be dissolved in suitable organic solvents, or a mixture of solvents, together with an emulsifying agent to enhance dispersion of the compound in water. The concentration of the total active ingredient in emulsifiable concentrates is usually from 10% to 90%, and in flowable emulsion concentrates, can be as high as 75%. As used herein, the term “active ingredient” refers to the total fungicidal composition, that is the combined quantity of pyridazinone and fatty acid used for a synergistic fungitoxic effect.

Wettable, powdered formulations suitable for spraying can be prepared by admixing the fungicidal compound with a finely divided solid, such as clays, inorganic silicates and carbonates, and silicas and incorporating wetting agents, sticking agents, and or dispersing agents in such mixtures. The concentration of total active ingredients in such formulations is usually in the range of from 20% to 99% by weight, preferably from 40% to 75%. A typical wettable powder is made by blending 50 parts of a pyridazinone, 45 parts of a synthetic precipitated hydrated silicon dioxide, such as that sold under the trademark Hi-SilR, and 5 parts of sodium lignosulfonate. To prepare a wettable powder from the compounds of the present invention, 50 parts of total active ingredients (pyridazinone and fatty acid, salt, or ester) may be used instead of 50 parts of pyridazinone. In another preparation a kaolin type (Barden) clay is used in place of the Hi-Sil in the above wettable powder, and in another such preparation 25% of the Hi-Sil is replaced with a synthetic sodium silicoaluminate sold under the trademark Zeolex.®.7 (J. M. Huber Corporation).

Dusting formulations are prepared by mixing the fungicidal compounds with finely divided inert solids which can be organic or inorganic in nature. Materials useful for this purpose include botanical flours, silicas, silicates, carbonates and clays. One convenient method of preparing a dust is to dilute a wettable powder with a finely divided carrier. Dust formulations or concentrates containing from 20% to 80% of the active ingredient are commonly made and are subsequently diluted to from 1% to 10% use concentration.

The fungicidal compound and formulations may be applied as fungicidal sprays by methods commonly employed, such as conventional high-gallonage hydraulic sprays, low-gallonage sprays, air-blast spray, aerial sprays and dusts. The dilution and rate of application will depend upon the type of equipment employed, the method of application, plants to be treated and diseases to be controlled. Generally, the compounds of this invention will be applied in an amount of from 0.06 to 60 kilograms (kg) per hectare and preferably from 1 to 28 kg per hectare of the active ingredient.

As a seed protectant, the fungicidal formulation may be coated on the seed. The usual dosage rate is from 0.05 ounce of “active ingredient” per hundred pounds of seed, to 20 ounces per hundred pounds of seed, preferably from 0.05 to 4 ounces per hundred pounds of seed, and more preferably from 0.1 to 1 ounce per hundred pounds of seed. As a soil fungicide the fungicidal formulation may be incorporated in the soil or applied to the surface usually at a rate of from 0.02 to 20, preferably from 0.05 to 10, and more preferably from 0.1 to 5 kg per hectare. As a foliar fungicide, the fungicidal formulation may be applied to growing plants at a rate of from 0.01 to 10 kg per hectare, preferably from 0.02 to 6 kg per hectare, and more preferably from 0.3 to 1.5 kg per hectare.

The fungicidal compounds (Δ-9 fatty acid desaturase inhibitors) of the present invention may be combined with other known fungicides Δ-9 fatty acid desaturase inhibitors to provide broad spectrum activity. Suitable fungicides for use in combination with the fungicidal compounds of the present invention include, but are not limited to, those compounds listed in U.S. Pat. No. 5,252,594 (see in particular columns 14 and 15).

Fungicidal formulations for mammalian use are prepared as described in U.S. Pat. Nos. 6,107,316 and 6,140,362. The method of the present invention may be used to treat diseases caused by fungi on or in animals, including humans, domestic animals such as cattle, pigs and poultry. A pharmaceutically acceptable amount of the fungicidal formulation is administered to animals by any means appropriate to the condition being treated.

For pharmaceutical use, the compounds described herein may be taken up in pharmaceutically acceptable carriers, such as, for example, solutions, suspensions, tablets, capsules, ointments, elixirs and injectable compositions. Pharmaceutical preparations may contain from 0.1% to 99% by weight of active ingredient. Preparations which are in single dose form, “unit dosage form”, preferably contain from 20% to 90% active ingredient, and preparations which are not in single dose form preferably contain from 5% to 20% active ingredient. As used herein, the term “active ingredient” refers to compounds described herein, salts thereof, and mixtures of compounds described herein with other pharmaceutically active compounds. Dosage unit forms such as, for example, tablets or capsules, typically contain from about 0.05 to about 1.0 g of active ingredient.

Suitable means of administering the pharmaceutical preparations include oral, rectal, topical (including dermal, buccal and sublingual), vaginal, parenteral (including subcutaneous, intramuscular, intravenous, intradermal, intrathecal and epidural) and by naso-gastric tube. It will be understood by those skilled in the art that the preferred route of administration will depend upon the condition being treated and may vary with factors such as the condition of the recipient.

According to the method of the present invention, the effective compounds described herein may be administered alone or in conjunction with other pharmaceutically active compounds. It will be understood by those skilled in the art that pharmaceutically active compounds to be used in combination with the compounds described herein will be selected in order to avoid adverse effects on the recipient or undesirable interactions between the compounds. As used herein, the term “active ingredient” is meant to include compounds described herein when used alone or in combination with one or more additional pharmaceutically active compounds.

Fungicidal formulations for controlling growth on a substrate or applied to a loci are prepared as described in U.S. Pat. Nos. 5,292,763 and 5,468,759. Important applications of the fungicidal formulations of the present invention include but are not limited to: inhibiting the growth of fungi in aqueous paints and coatings, adhesives, sealants, latex emulsions, and joint cements; preserving wood; preserving cutting fluids, controlling slime-producing fungi in pulp and paper mills and cooling towers; as a spray or dip treatment for textiles and leather to prevent mold growth; protecting paint films, especially exterior paints, from attack by fungi which occurs during weathering of the paint film; protecting processing equipment from slime deposits during manufacture of cane and beet sugar; preventing microorganism buildup and deposits in air washer or scrubber systems and in industrial fresh water supply systems; preserving fuel; controlling microorganisms contamination and deposits in oil field drilling fluids and muds, and in secondary petroleum recovery processes; preventing fungal growth in paper coatings and coating processes; controlling fungal growth and deposits during the manufacture of various specialty boards, e.g., cardboard and particle board; preventing sap stain discoloration on freshly cut wood of various kinds; controlling fungal growth in clay and pigment slurries of various types; as a hard surface disinfectant to prevent growth of fungi on walls, floors, etc.; as a preservative for cosmetic and toiletry products, floor polishes, fabric softeners, household and industrial products, in swimming pools to prevent algae growth; inhibiting the growth of harmful yeasts and fungi on plants, trees, fruits, seeds, or soil; preserving agricultural formulations, electrodeposition systems, diagnostic and reagent products, medical devices; protecting animal dip compositions against the buildup of microorganisms, and in photo processing to prevent buildup of microorganisms, and the like.

The fungicidal compounds and formulations of the present invention also have biocidal applications including, but not limited to, wood preservatives, leather preservatives and marine anti-foulants. Accordingly, the present invention also encompasses the use of the disclosed compositions as wood preservatives and marine anti-foulants.

The invention is illustrated in the following examples.

### EXAMPLE 1

### Control of Candida Albicans Infection in Mice by Compound 1

Compound 1 (6-(4-chlorophenyl)-2-(2′-pentynyl)pyridazinone, described as compound 109 in European Patent Publication No. EP 478 195 A1) in 5% dimethylsulfoxide (DMSO) in saline solution was administered intraperitoneally 1 h before and at 4, 24, 48 and 72 h after intravenous inoculation with Candida albicans (American Type Culture Collection strain 10231). Ten mice were used for each treatment. Mortality was recorded over a period of 10 days.

As shown in Table 1, compound 1 was found to be an effective inhibitor of Candida albicans infection in mice.

TABLE 1
Control of Candida albicans infection in mice by compound 1

| Treatment | Dose mg/kg | % Survival |
|---|---|---|
| Vehicle control | | 10 |
| Compound 1 | 100 | 50 |
| | 30 | 40 |
| | 10 | 20 |
| Amphotericin B | 10 | 100 |

### EXAMPLE 2

### Effect of Compound 1 on Activity of Fungal and Mammalian Δ-9 Fatty Acid Desaturases

The following method was used to prepare the microsomal Δ-9 fatty acid desaturase from yeast. Two 250 ml flasks containing 100 ml each of glucose-yeast extract medium (0.4% yeast extract and 2% glucose) were inoculated with Saccharomyces cerevisiae strain 12341 obtained from the American Type Culture Collection, and grown for 48 h at 30° C. with shaking at 250 rpm. The cells were used to inoculate a 4 liter flask containing 3.6 liters of medium. The cells were grown at 30° C. with gentle shaking at 90 rpm for 24 h. The cells were centrifuged at 2,500×G for 5 min. at 4° C., the cell pellet washed twice by suspension in ice-cold water and re-centrifugation, then re-suspended in an equal volume of cold 0.1 M potassium phosphate buffer, pH 7.2. The cells were lysed by homogenizing in a French press at an output pressure of 18,000 psi, and the homogenate was centrifuged at 8,000×G for 20 min. at 4° C. The supernatant was filtered through a plug of glass wool to remove the floating lipid layer and then centrifuged at 100,000×G for 90 min. at 4° C. and the resulting microsomal pellet suspended in 10 ml of ice-cold 0.1 M potassium phosphate buffer, pH 7.2, using a Dounce homogenizer to give a protein concentration of approximately 10 mg/ml. The preparation was frozen as aliquots in dry-ice/methanol and stored at −80° C.

The following method was used to prepare the microsomal Δ-9 fatty acid desaturase from rat liver. Sprague-Dawley rats weighing between 120 and 180 g were fed with Purina laboratory chow, starved for 48 h, then fed with a “Fat-Free” test diet (Nutritional Biochemical Corporation) for 20 h. Livers were removed and placed in cold 10 mm Tris-acetate buffer, pH 8.1, containing 0.25 M sucrose and 1 mM EDTA. The tissue was washed twice with 10 volumes of cold buffer, blotted dry and weighed. The tissue was minced and homogenized in 5 ml of buffer per gram of tissue using a glass homogenizer with a loose fitting Teflon pestle. The homogenate was centrifuged twice at 18,000×G for 15 min., discarding the pellet after each centrifugation. The supernatant was centrifuged at 120,000×G for 30 min to obtain a pellet with two layers. The upper gelatinous layer is the microsomal fraction while the lower layer consists of glycogen. The microsomal layer was carefully re-suspended in 0.1 M Tris-acetate buffer, pH 8.1, containing 0.5 M NaCl and centrifuged again at 120,000 g for 30 min. The pellet was re-suspended in 0.1 M Tris-acetate buffer, pH 8.1, using a Dounce homogenizer to give a protein concentration of approximately 10 mg/ml, and frozen as aliquots in dry-ice/methanol and stored at −80° C.

Delta-9 Fatty acid desaturase assays were performed by measuring the desaturation of ¹⁴C-palmitoyl-CoA to palmitoleic acid using 0.5 ml reaction mixtures containing 0.1 M potassium phosphate buffer, pH 7.2, 1 mM NADH and 26 μM ¹⁴C-palmitoyl-CoA (0.028 μCi per assay) in 13×100 mm glass culture tubes. Compound 1 was added as 5 μl of solution in dimethylsulfoxide (DMSO), and tested at a series of two-fold dilutions. The reagents were added to the culture tubes on ice, and the microsomal enzyme preparation (0.2 mg protein) added last. The tubes were incubated with vigorous shaking at 200 rpm at 30° C. for 5 min., then the reaction was stopped by adding 0.5 ml of 10% potassium hydroxide in methanol/water (90:10, v/v). The tubes were capped and saponified by heating at 80° C. for 30 min. 6 M HCl (0.5 ml) was added to each tube, followed by 0.5 ml of cyclohexane. The tubes were mixed vigorously and centrifuged briefly at 2,000 rpm to facilitate phase separation. The upper cyclohexane layer was removed and 100 μI analyzed by HPLC on a Supelcosil LC-18 column (25×4.6 mm) with methanol-water-phosphoric acid (90:9.9:0.1, by volume) as the mobile phase at a flow rate of 1 ml/min. A Packard A120 RAM detector was used to determine the amounts of radioactivity in the palmitic and palmitoleic acid fractions. The percent inhibition of desaturation was determined by comparing the production of palmitoleic acid in assays containing compound 1 with production in assays containing DMSO alone. The concentration of compound 1 which inhibited production of palmitoleic acid by 50% was determined from dose-response curves.

The data in Table 2 clearly demonstrate that compound 1 is a potent inhibitor of fungal Δ-9 fatty acid desaturase but does not inhibit mammalian Δ-9 fatty acid desaturase.

TABLE 2
Potency of compound 1 towards yeast and
rat liver Δ-9 fatty acid desaturases.

| Yeast | Rat liver |
|---|---|
| desaturase | desaturase |
| EC50 (ppm) | EC50 (ppm) |
| 0.055 | >100 |

### EXAMPLE 3

### Effect of Fatty Acids on Fungitoxicity of Compound 1 and Cerulenin

Fifty ml of YPG culture medium (10 g yeast extract, 20 g peptone, and 20 g glucose per liter of water) in a 250 ml Erlenmeyer flask was inoculated with Saccharomyces cereviae (strain X2180-1A) to give an absorbance at 700 nm of 0.02. This culture was incubated for 18 h at 30° C. with shaking at 225 rpm. The cells were harvested by centrifugation, washed once with SD medium (6.7 g Bacto-yeast nitrogen base and 20 g glucose per liter of water), and suspended in SD medium to give an Absorbance at 700 nm of 0.04. Aliquots (5 ml) of cell suspension were distributed in 20 ml capacity glass vials. Treatments containing compound 1 or cerulenin received 25 μl of a solution of compound in dimethylsulfoxide (DMSO) at 240 ppm or 160 ppm, respectively. Control treatments without compound 1 or cerulenin received 25 ul DMSO. Treatments containing a fatty acid received 25 μl of a 2 mM solution of the fatty acid in a 1% solution of Triton X-100 providing a concentration of 10 μM. Treatments without fatty acid received 25 ul of 1% Triton X-100. All treatments were carried out in duplicate. Vials were incubated for 22 h at 30° C. with shaking at 225 rpm. Growth of the cells was then estimated by diluting the cells in each treatment 3-fold with SD medium and measuring the asorbance at 700 nm. The results in Table 3 show inhibition of growth expressed as a percentage of growth in DMSO controls containing Triton X-100 but lacking Compound 1, cerulenin or fatty acid.

The data in Table 3 illustrate that the fungitoxicity of compound 1 is reduced by unsaturated fatty acids (oleic and palmitoleic acids) but not by saturated fatty acids (pentadecanoic and palmitic acids). In contrast, the fungitoxicity of cerulenin, which inhibits another enzyme involved in fatty acid biosynthesis called fatty acid synthetase, is reduced by both unsaturated and saturated fatty acids. The inventor demonstrates the ability to identify inhibitors of Δ-9 fatty acid desaturase by screening for compounds which show fungitoxicity which is reduced in the presence of unsaturated fatty acids but not in the presence of saturated fatty acids. Furthermore inhibitors of Δ-9 fatty acid desaturase can be distinguished from inhibitors of other enzymes involved in fatty acid biosynthesis by this approach.

TABLE 3
Effect of fatty acids on fungitoxicity of
compound 1 and cerulenin.

| | % Inhibition | % Inhibition |
|---|---|---|
| | Compound 1 | Cerulenin |
| Fatty acid | (1.2 ppm) | (0.8 ppm) |
| None | 76.9 | 35.6 |
| oleic acid | 18.7 | 3.3 |
| palmitoleic | 21.8 | 4.5 |
| palmitic | 95.5 | 15.2 |
| pentadecanoic | 96.7 | 20.3 |

### EXAMPLE 4

### Analysis of the Fatty Acid Composition of Saccharomyces cerevisiae after Treatment with Compound 1

S. cerevisiae was grown in SD medium as 5 ml aliquots of cell suspension in 20 ml capacity glass vials as described in example 3 in the absence (controls) and presence of compound 1. Compound 1 was added as 25 μl of solution in DMSO, and controls received 25 ul DMSO alone. After growth for 18 h at 30° C. with shaking at 225 rpm, inhibition of growth by compound 1 was determined as described in example 3, and the fatty acid composition was determined as follows. The cells were centrifuged in 20 ml borosilicate glass culture tubes at 2000 rpm. The supernatant was decanted, and the cells were re-suspended in 10 ml of water and re-centrifuged at 2000 rpm. The supernatant was decanted, and the cells were saponified by heating at 80° C. for 1 hour in 2.5 ml of 10% potassium hydroxide solution in 90% methanol/10% water. The non-saponifiable lipids were removed by extracting the basic solution with 2×3 ml of cyclohexane. The basic solution was then acidified to pH 2 with 6N hydrochloric acid, and the acidic solution was again extracted with 2×3 ml of cyclohexane. These extracts were analyzed directly by capillary gas chromatography using an HP5890 gas chromatograph equipped with a Restek Stabilwax DB capillary column (30m×0.53 mm i.d.×0.25 μm film thickness) and a flame ionization detector. Analyses were run at 225° C. isothermally, and compared to a sample free fatty acid mixture prepared from authentic standards purchased from Sigma. The results, as shown in Table 4, show that levels of the unsaturated fatty acids (palmitoleic and oleic acids) were greatly reduced whereas levels of the saturated fatty acids (palmitic and stearic) are relatively unaffected.

TABLE 4
Effects of compound 1 on the fatty acid composition
and growth of S. cerevisiae.

| Compound 1 | Growth % | palmitic | palmitoleic | stearic | oleic |
|---|---|---|---|---|---|
| μg/ml | Inhibition | μg/mg^{a} | μg/mg | μg/mg | μg/mg |
| 0 | 0 | 6.1 | 16.8 | 2.3 | 9.8 |
| 0.0625 | 10.1 | 7.1 | 14.2 | 2.8 | 8.5 |
| 0.125 | 14.3 | 6.5 | 14 | 3.1 | 4.3 |
| 0.25 | 19.1 | 6 | 3 | 3 | 2.7 |
| 0.5 | 38.7 | 5.2 | 1 | 4.7 | 1.1 |
| 1 | 54.4 | 6.7 | 0 | 5 | 1.2 |
| 2 | 66.4 | 5 | 0 | 4.2 | 0.5 |

^{a}micrograms of fatty acid per milligram dry weight of cells

1. A method for identifying potential fungitoxic compounds which comprises (a) evaluating candidate compounds for the ability to bind to a Δ-9 fatty acid desaturase enzyme, and (b) measuring a significant reduction of enzyme activity.

2. The method according to claim 1 wherein the compounds that inhibit the Δ-9 fatty acid desaturase enzyme are subjected to one or more tests to confirm fungicidal activity.

3. The method according to claim 1 which includes assaying the activity of Δ-9 fatty acid desaturase in a microsomal preparation from fungi in the absence and presence of candidate compounds.

4. The method according to claim 1 wherein activity of Δ-9 fatty acid desaturase is determined by measuring the conversion of a saturated fatty acyl-coenzyme A substrate to an unsaturated fatty acid product.

5. The method according to claim 1 wherein the fungitoxic compound is effective against classes of fungi selected from the group consisting of Ascomycete, Basidiomycete, Deuteromycete, Oomycete and combinations thereof.

6. The method according to claim 1 wherein the assay detects the inhibition of Δ-9 fatty acid desaturase activity in whole cells or cell extracts from plant pathogenic fungi selected from the group consisting of Colletotrichum spp., Magnaporthe spp., Botrytis spp., Fusarium spp., Alternaria spp., Helminthosporium spp., Venturia spp., Cercospora spp., Septoria spp., Mycosphaerella spp., Monilinia spp., Sclerotinia spp., Puccinia spp., Phytophthora spp., Pythium spp., Erysiphe spp., Penicillium spp. and Puccinia spp. and combinations thereof

7. The method according to claim 1 wherein the assay detects the inhibition of Δ-9 fatty acid desaturase activity in whole cells or cell extracts from a fungal pathogen of mammals selected from the group consisting of Candida spp., Aspergillus spp., Fusarium spp., Coccidioides immitis, Cryptococcus neoformans, Histoplasma capsulatum, Microsporum spp., and Tricophyton spp.

8. The method according to claim 1 wherein the assay detects the inhibition of Δ-9 fatty acid desaturase in whole cells or cell extracts from Saccharomyces cerevisiae or other fungi which may serve as suitable model systems for plant or mammalian fungal pathogens.

9. The method according to claim 1 wherein compounds determined to inhibit Δ-9 fatty acid desaturase enzyme activity are used to prepare fungitoxic compositions and formulations.

10. A method for identifying fungitoxic compounds which comprises (a) screening at least one candidate compound for fungitoxic in the presence and absence of an exogenous supply of (i) an unsaturated fatty acid and (ii) a saturated fatty acid; and (b) identifying inhibitors of Δ-9 fatty acid desaturase activity by demonstrating reduced fungitoxicity in the presence of the exogenous supply of unsaturated fatty acid and no reduction in fungitoxicity in the presence of the exogenous supply of saturated fatty acid.

11. A method for controlling fungal growth on a plant, animal or substrate which comprises testing candidate compounds in an assay which detects the inhibition of Δ-9 fatty acid desaturase activity and applying compounds which inhibit Δ-9 fatty acid desaturase activity to a locus on the plant, animal or substrate.

12. The method according to claim 11 wherein the substrate is selected from the group consisting of wood, leather, concrete, paints, plastics, metals and surfaces having a protective coating.

13. A fungitoxic composition comprising at least one A-9 fatty acid desaturase inhibitor as the active ingredient in combination with one or more carriers.

14. The fungitoxic composition of claim 13 wherein the composition includes one or more saturated fatty acids selected from the group consisting of fatty acids, fatty esters, fatty ethers and compounds having a formula CH3 (CH2)nCO2 X, wherein X is selected from H, alkali metals and C1-C8 alkyl and wherein n is an integer selected from 8-22.

15. The fungitoxic composition of claim 13 excluding pyridazinones of the type selected from the group consisting of 6-(4-chlorophenyl)-2-(2′-pentyn-4′-ene-1-yl)-3(2H)-pyridazinone; 6-(4-chlorophenyl)-2-(2′-pentynyl)-3(2H)-pyridazinone; 6-(4-chlorophenyl)-2-(5′-pentoxy-2′-butynyl)-3(2H)-pyridazi-none; 6-(4-chloro-phenyl)-2-(4′-fluoro-2′-butynyl)-3(2H)-pyridazinone; 6-(2-pyridyl)-2-(2′-nonynyl)-3(2H)-pyridazinone; 7-chloro-2,4,4a,5-tetrahydro-2-(2′-pentyn-yl)-indeno[1,2-c]-pyridazin-3-one; 6-(4-chlorophenyl)-2-(2′-pentynyl)-3(2H)-4,5-dihydropyridazinone; 6-(2-napthyl)-2-(2′-pentynyl)-3(2H)-pyridazi-none; 6-(4-chlorophenyl)-2-(2′-decynyl)-3(2H)-pyridazinone and combinations thereof.

16. The fungitoxic composition of claim 13 wherein the carrier is selected from organic solvents selected from the group consisting of water, acetone, methanol, ethanol, dimethylformamide, pyridine and dimethyl sulfoxide and finely divided solids selected from the group consisting of clays, inorganic silicates and carbonates, and silicas.

17. A fungitoxic formulation comprising; (a) at least one Δ-9 fatty acid desaturase inhibitor (b) at least one fungicide that is not a Δ-9 fatty acid desaturase inhibitor; (c) optionally one or more carriers; and (d) optionally one or more additives or stabilizers.

18. The fungitoxic formulation according to claim 17 wherein the formulation includes one or more saturated fatty acids selected from the group consisting of fatty acids, fatty esters, fatty ethers and compounds having a formula CH3 (CH2)nCO2 X, wherein X is selected from H, alkali metals and C1-C8 alkyl and wherein n is an integer selected from 8-22.

19. A method for controlling growth of fungi on plants, mammals or substrates which comprises testing candidate compounds in an assay which detects the inhibition of Δ-9 fatty acid desaturase activity and applying the fungitoxic formulation of claim 18 to a locus wherein control of fungal growth is desired.

20. The method according to claim 19 wherein the substrate is selected from the group consisting of paint, wood, leather, concrete, metal, plastics and animal skin.
